(19) United States
(12) Patent Application Publication
(10) Pub. No.: US 2007/0248961 A1
(43) Pub. Date: Oct. 25, 2007

(54) Methods for detecting mutations in JAK2 nucleic acid

(76) Inventors: Maher Albitar, Coto De Caza, CA (US); Wanglong Ma, Aliso Viejo, CA (US)

Correspondence Address: FOLEY & LARDNER LLP, P.O. BOX 80278, SAN DIEGO, CA, 92138-0278, US

(21) Appl. No.: 11/408,241

(22) Filed: Apr. 20, 2006

(51) Int. Cl. C12Q 1/68

(52) U.S. Cl. 435/6

(57) ABSTRACT

The present invention relates to methods for detecting JAK2 nucleic acid in acellular bodily fluid samples from patients with neoplastic disease and determining if the nucleic acid contains one or more mutations or one mutation and one deletion. The methods are useful for diagnosing patients that have cells with mutations in the JAK2 gene that effect kinase activity. The detection of such mutations can be used to determine treatment for patients or stratifying patients for therapy and management.

### FIELD OF THE INVENTION

This invention relates to the field of cancer detection and more specifically to diagnostic methods useful for patients having neoplastic disease such as a myeloproliferative disease.

### BACKGROUND OF THE INVENTION

The following discussion of the background of the invention is merely provided to aid the reader in understanding the invention and is not admitted to describe or constitute prior art to the invention.

Certain neoplastic diseases including non-CML myeloproliferative diseases (MPDs) such as polycythemia vera (PV), essential thrombocythemia (ET), and chronic idiopathic myelofibrosis (IMF) and as of yet unclassified myeloproliferative diseases (MPD-NC) are characterized by an aberrant increase in blood cells. See e.g., Vainchenker and Constantinescu, Hematology (American Society of Hematology), 195-200 (2005). This increase is generally initiated by a spontaneous mutation in a multipotent hematopoetic stem cell located in the bone marrow. Id. Due to the mutation, the stem cell produces far more blood cells of a particular lineage than normal, resulting in the overproduction of cells such as erythroid cells, megakaryocytes, granulocytes and monocytes. Some symptoms common to patients with MPD include enlarged spleen, enlarged liver, elevated white, red and/or platelet cell count, blood clots (thrombosis), weakness, dizziness and headache. Diseases such as PV, ET and IMF may presage leukemia, however the rate of transformation (e.g., to blast crisis) differs with each disease. Id.

The specific gene and concomitant mutation or mutations responsible for many MPDs is not known. However, a mutation in the Janus kinase 2 (JAK2) gene, a cytoplasmic, nonreceptor tyrosine kinase, has been identified in a number of MPDs. For example, this mutation has been reported in up to 97% of patients with PV, and in greater than 40% of patients with either ET or IMF. See e.g., Baxter et al., Lancet 365:1054-1060 (2005); James et al., Nature 438:1144-1148 (2005); Zhao, et al., J. Biol. Chem. 280(24):22788-22792 (2005); Levine et al., Cancer Cell, 7:387-397 (2005); Kralovics, et al., New Eng. J. Med. 352(17):1779-1790 (2005); Jones, et al., Blood 106:2162-2168 (2005); Steensma, et al., Blood 106:1207-2109 (2005).

The Janus kinases are a family of tyrosine kinases that play a role in cytokine signaling. For example, JAK2 kinase acts as an intermediary between membrane-bound cytokine receptors such as the erythropoietin receptor (EpoR), and down-stream members of the signal transduction pathway such as STAT5 (Signal Transducers and Activators of Transcription protein 5). See, e.g, Schindler, C. W., J. Clin Invest. 109:1133-1137 (2002); Tefferi and Gilliland, Mayo Clin. Proc. 80:947-958 (2005); Giordanetto and Kroemer, Protein Engineering, 15(9):727-737 (2002). JAK2 is activated when cytokine receptor/ligand complexes phosphorylate the associated JAK2 kinase. Id. JAK2 can then phosphorylate and activate its substrate molecule, for example STAT5, which enters the nucleus and interacts with other regulatory proteins to affect transcription. Id.; Nelson, M. E., and Steensma, D. P., Leuk. Lymphoma 47:177-194 (2006).

In the JAK2 mutant, a valine (codon “GTC”) is replaced by a phenylalanine (codon “TTC”) at amino acid position 617 (the “V617F mutant”). Baxter et al., Lancet 365:1054-1060 (2005). Amino acid 617 is located in exon 12 which includes a pseudokinase, auto-inhibitory (or negative regulatory) domain termed JH2 (Jak Homology 2 domain). Id.; James et al., Nature 438:1144-1148 (2005). Though this domain has no kinase activity, it interacts with the JH1 (Jak Homology 1) domain, which does have kinase activity. Baxter et al., Lancet 365:1054-1060 (2005). Appropriate contact between the two domains in the wild-type protein allows proper kinase activity and regulation; however, the V617F mutation causes improper contact between the two domains, resulting in constitutive kinase activity in the mutant JAK2 protein. Id.

A variety of different approaches and a large body of evidence suggest that, when present, the JAK2 V617F mutation contributes to the pathogenesis of MPD. See e.g., Kaushansky, Hematology (Am Soc Hematol Educ Program), 533-7 (2005). The mutation has been detected from blood samples, bone marrow and buccal samples (see, e.g, Baxter et al., Lancet 365:1054-1060 (2005); James et al., Nature 438:1144-1148 (2005); Zhao, et al., J. Biol. Chem. 280(24):22788-22792 (2005); Levine et al., Cancer Cell, 7:387-397 (2005); Kralovics, et al., New Eng. J. Med. 352(17):1779-1790 (2005)), and homozygous and heterozygous cell populations have been reported in MPD patients. Baxter et al., Lancet 365:1054-1060 (2005).

Here we demonstrate that the presence or absence of the JAK2 V617F mutation and the zygosity status (e.g., wild-type, homozygous/hemizygous or heterozygous) confer differences in survival and longevity in some patient populations. However, the characterization of the zygosity status of cell populations from samples such blood cells, bone marrow cells or buccal cells using standard detection methods is difficult because the wild-type JAK2 sequences from normal cells are detected along with any mutants, and samples that may contain homozygous or hemizygous cell populations appear heterozygous.

Accordingly, there is a need in the art for methods to more easily and accurately identify patients carrying the mutation, and for methods to characterize patient cell populations as homozygous, hemizygous/heterozygous or wild-type for the JAK2 V617F mutation.

### SUMMARY OF THE INVENTION

The invention relates to methods for detecting and characterizing nucleic acid in patient samples. In particular aspects, the invention relates to determining the presence or absence of JAK2 mutations in RNA from acellular bodily fluids of patients with neoplastic disease.

In one aspect, the invention provides a method for determining the presence or absence of one or more mutations in JAK2 nucleic acid from an acellular bodily fluid of a patient. In a related aspect, the invention provides a method for treatment of a patient with neoplastic disease which includes determining the presence or absence of one more mutations in JAK2 nucleic acid from an acellular bodily fluid of the patient and treating the patient based on the determination. In another aspect, the invention provides a method for determining whether a patient diagnosed with a neoplastic disease has cells containing JAK2 mutant kinase activity which includes determining the presence or absence of one or more mutations in JAK2 nucleic acid from an acellular bodily fluid of the patient. In other aspects, the invention provides a method for diagnosing a neoplastic disease which includes determining the presence or absence of one or more mutations in JAK2 nucleic acid from an acellular bodily fluid of a patient. In another aspect, the invention provides a method of determining a prognosis of an individual diagnosed with a neoplastic disesase such as polycythemia vera, essential thrombocythemia, idiopathic myelofibrosis, or unclassified myeloproliferative disease, comprising determining the presence or absence of one or more mutations in JAK2 nucleic acid in an acellular bodily fluid of the individual and using the mutation status to predict the clinical outcome for the individual.

In preferred embodiments of the above aspects of the invention, the presence or absence of one or more mutations may be determined relative to SEQ ID NO: 1 or SEQ ID NO: 2. In other preferred embodiments, one or more mutations affects kinase activity; more preferably, one or more mutations are located in an activation domain or more specifically in a pseudokinase domain of JAK2; preferably at least one of the mutations is at codon 617; preferably, the mutation codes for an amino acid other than valine; more preferably the mutation causes a V671F amino acid change.

In certain preferred embodiments, the patient has been diagnosed with a myeloproliferative disease; more preferably, the patient has been diagnosed with polycythemia vera, essential thrombocythemia, idiopathic myelofibrisis, or an unclassified myeloproliferative disease.

In other preferred embodiments, the acellular bodily fluid is plasma or serum. In some embodiments, the JAK2 nucleic acid is RNA.

In still other preferred embodiments of the methods of the invention, determining the presence or absence of one or more mutations includes reverse transcribing the JAK2 nucleic acid; more preferably determining the presence or absence of one or more mutations includes amplifying JAK2 nucleic acid; preferably, determining the presence or absence of one or more mutations includes reverse transcribing the JAK2 nucleic acid and amplifying; more preferably, determining the presence or absence of one or more mutations includes amplifying JAK2 nucleic acid and hybridizing the amplified nucleic acid with an oligonucleotide probe that is capable of specifically detecting JAK2 nucleic acid under hybridization conditions; in other preferred embodiments, determining the presence or absence of one or more mutations includes amplifying JAK2 nucleic acid and sequencing the amplified nucleic acid.

In certain preferred embodiments, the methods of the invention also include determining if an acellular bodily fluid of a patient contains mutant JAK2 nucleic acid and wild-type JAK2 nucleic acid; preferably, a ratio of mutant JAK2 nucleic acid relative to wild-type JAK2 nucleic acid is determined; preferably a diagnosis or treatment is based on one or more of the determinations; preferably a treatment is administered, foregone or changed based on one or more of the determinations.

In another aspect, the invention provides methods for determining a prognosis of an individual diagnosed with a neoplastic disease such as polycythemia vera, essential thrombocythemia, idiopathic myelofibrosis, or unclassified myeloproliferative disease, the method comprising determining the presence or absence of one or more mutations in JAK2 nucleic acid in an acellular bodily fluid of the individual and using the mutation status to predict the clinical outcome for the individual. In some assay methods, the proportion of wild-type to mutant JAK2 nucleic acid present in the sample is determined to make a prognosis. In some methods, the patient sample may contain no or a minimal amount of JAK2 mutant nucleic acid relative to wild-type JAK2 nucleic acid, or the sample may contain no or a minimal amount of JAK2 wild-type nucleic acid relative to mutant JAK2 nucleic acid in the sample. In some cases, the mutation status is hemizygous or homozygous mutant for JAK2. The clinical outcome may be death. In some embodiments, the mutation status is combined with other clinical parameters to determine the clinical outcome for the individual. For example, the other clinical parameters may be the age of the individual or the precent blast cell count.

The term “neoplastic disease” refers to a condition characterized by an abnormal growth of new cells such as a tumor. A neoplasm includes solid and non-solid tumor types such as a carcinoma, sarcoma, leukemia and the like. A neoplastic disease may be malignant or benign.

The term “myeloproliferative disease” or “myeloproliferative disorder” is meant to include non-lymphoid dysplastic or neoplastic conditions arising from a haematopoietic stem cell or its progeny. “MPD patient” includes a patient who has been diagnosed with an MPD. “Myeloproliferative disease” is meant to encompass the specific, classified types of myeloproliferative diseases including polycythemia vera (PV), essential thrombocythemia (ET) and idiopathic myelofibrosis (IMF). Also included in the definition are hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, and systemic mastocytosis (SM). “Myeloproliferative disease” is also meant to encompass any unclassified myeloproliferative diseases (UMPD or MPD-NC).

As used herein, the term “patient” refers to one who receives medical care, attention or treatment. As used herein, the term is meant to encompass a person diagnosed with a disease such a myeloproliferative disease as well as a person who may be symptomatic for a disease but who has not yet been diagnosed.

The term “sample” or “patient sample” is meant to include biological samples such as tissues and bodily fluids. “Bodily fluids” may include, but are not limited to, blood, serum, plasma, saliva, cerebral spinal fluid, pleural fluid, tears, lactal duct fluid, lymph, sputum, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin.

As used herein, “plasma” refers to acellular fluid found in blood. “Plasma” may be obtained from blood by removing whole cellular material from blood by methods known in the art (e.g., centrifugation, filtration, and the like). As used herein, “peripheral blood plasma” refers to plasma obtained from peripheral blood samples.

As used herein, “serum” includes the fraction of plasma obtained after plasma or blood is permitted to clot and the clotted fraction is removed.

The term “nucleic acid” or “nucleic acid sequence” refers to an oligonucleotide, nucleotide or polynucleotide, and fragments or portions thereof, which may be single or double stranded, and represent the sense or antisense strand. A nucleic acid may include DNA or RNA, and may be of natural or synthetic origin. For example, a nucleic acid may include mRNA or cDNA. Nucleic acid may include nucleic acid that has been amplified (e.g., using polymerase chain reaction).

The term “source of nucleic acid” refers to any sample which contains nucleic acids (RNA or DNA). Particularly preferred sources of target nucleic acids are biological samples including, but not limited to blood, plasma, serum, saliva, cerebral spinal fluid, pleural fluid, milk, lymph, sputum and semen.

An “amino acid sequence” refers to a polypeptide or protein sequence. The convention “AA_{wt}###AAₘᵤₜ” is used to indicate a mutation that results in the wild-type amino acid AA_{wt} at position ### in the polypeptide being replaced with mutant AAₘᵤₜ.

A “gene” refers to a DNA sequence that comprises control and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. “Wild-type” may also refer to the sequence at a specific nucleotide position or positions, or the sequence at a particular codon position or positions, or the sequence at a particular amino acid position or positions. For example, a gene can be “wild-type” at nucleotide position 1849 or at codon 617. As used herein, “mutant,” “modified” or “polymorphic” refers to a gene or gene product which displays modifications in sequence and or functional properties (i.e., altered characteristics) when compared to the wild-type gene or gene product. “Mutant,” “modified” or “polymorphic” also refers to the sequence at a specific nucleotide position or positions, or the sequence at a particular codon position or positions, or the sequence at a particular amino acid position or positions.

A “mutation” is meant to encompass at least a single nucleotide variation in a nucleic acid sequence relative to the normal sequence or wild-type sequence. A mutation may include a substitution, a deletion, an inversion or an insertion. With respect to an encoded polypeptide, a mutation may be “silent” and result in no change in the encoded polypeptide sequence or a mutation may result in a change in the encoded polypeptide sequence. For example, a mutation may result in a substitution in the encoded polypeptide sequence. A mutation may result in a frameshift with respect to the encoded polypeptide sequence.

As used herein the term “codon” refers to a sequence of three adjacent nucleotides (either RNA or DNA) constituting the genetic code that determines the insertion of a specific amino acid in a polypeptide chain during protein synthesis or the signal to stop protein synthesis. The term “codon” is also used to refer to the corresponding (and complementary) sequences of three nucleotides in the messenger RNA into which the original DNA is transcribed.

The term “homology” or “homologous” refers to a degree of identity. There may be partial homology or complete homology. A partially homologous sequence is one that has less than 100% sequence identity when compared to another sequence.

“Heterozygous” refers to having different alleles at one or more genetic loci in homologous chromosome segments. As used herein “heterozygous” may also refer to a sample, a cell, a cell population or an organism in which different alleles at one or more genetic loci may be detected. Heterozygous samples may also be determined via methods known in the art such as, for example, nucleic acid sequencing. For example, if a sequencing electropherogram shows two peaks at a single locus and both peaks are roughly the same size, the sample may be characterized as heterozygous. Or, if one peak is smaller than another, but is at least about 25% the size of the larger peak, the sample may be characterized as heterozygous. In some embodiments, the smaller peak is at least about 15% of the larger peak. In other embodiments, the smaller peak is at least about 10% of the larger peak. In other embodiments, the smaller peak is at least about 5% of the larger peak. In other embodiments, a minimal amount of the smaller peak is detected.

As used herein, “homozygous” refers to having identical alleles at one or more genetic loci in homologous chromosome segments. “Homozygous” may also refer to a sample, a cell, a cell population or an organism in which the same alleles at one or more genetic loci may be detected. Homozygous samples may be determined via methods known in the art, such as, for example, nucleic acid sequencing. For example, if a sequencing electropherogram shows a single peak at a particular locus, the sample may be termed “homozygous” with respect to that locus.

The term “hemizygous” refers to a gene or gene segment being present only once in the genotype of a cell or an organism because the second allele is deleted. As used herein “hemizygous” may also refer to a sample, a cell, a cell population or an organism in which a allele at one or more genetic loci may be detected only once in the genotype.

The term “zygosity status” as used herein refers to a sample, a cell population, or an organism as appearing heterozygous, homozygous, or hemizygous as determined by testing methods known in the art and described herein. The term “zygosity status of a nucleic acid” means determining whether the source of nucleic acid appears heterozygous, homozygous, or hemizygous. The “zygosity status” may refer to differences in a single nucleotide in a sequence. In some methods, the zygosity status of a sample with respect to a single mutation may be categorized as homozygous wild-type, heterozygous (i.e., one wild-type allele and one mutant allele), homozygous mutant, or hemizygous (i.e., a single copy of either the wild-type or mutant allele). Because direct sequencing of plasma or cell samples as routinely performed in clinical laboratories does not reliably distinguish between hemizygosity and homozygosity, in some embodiments, these classes are grouped. For example, samples in which no or a minimal amount of wild-type nucleic acid is detected are termed “hemizygous/homozygous mutant.” In preferred embodiments, a “minimal amount” may be between about 1-2%. In other embodiments, a minimal amount may be between about 1-3%. In still other embodiments, a “minimal amount” may be less than 1%.

The term “substantially all” means between about 60-100%, more preferably, between about 70-100%; more preferably between about 80-100%, more preferably between about 90-100%, and more preferably between about 95-100%.

An oligonucleotide (e.g., a probe or a primer) that is specific for a target nucleic acid will “hybridize” to the target nucleic acid under suitable conditions. As used herein, “hybridization” or “hybridizing” refers to the process by which a oligonucleotide single strand anneals with a complementary strand through base pairing under defined hybridization conditions.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art.

Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

Where the detected JAK2 nucleic acid is a fragment or portion of a full-length JAK2 nucleic acid sequence, the fragment or portion may include at least about 60% of the pseudokinase domain; preferably, the fragment or portion may include about 70% of the pseudokinase domain; more preferably, the fragment or portion may include about 80% of the pseudokinase, preferably about 90%-95% of the pseudokinase domain. The JAK2 nucleic acid may encode a polypeptide having kinase activity (i.e., tyrosine kinase activity), or may include a polypeptide having no kinase activity.

For the JAK2 nucleic acid sequence, a “mutation” means a JAK2 nucleic acid sequence that includes at least one nucleic acid variation as compared to reference sequence GenBank accession number NM004972 (SEQ ID NO: 1, FIG. 1), or SEQ ID NO:2, FIG. 2. A mutation may include a substitution, a deletion or an insertion. A mutation in JAK2 nucleic acid may result in a change in the encoded polypeptide sequence or the mutation may be silent with respect to the encoded polypeptide sequence. An example of a JAK2 mutation that results in a change in polypeptide sequence includes, but is not limited to V617F. A change in an amino acid sequence may be determined as compared to SEQ ID NO: 3, FIG. 3 as a reference amino acid sequence.

The term “determined relative to” in reference to determining the presence or absence of one or more mutations in JAK2 mutations has the same meaning as the term “compared to.”

“Determining the presence or absence of one or more mutations” in a nucleic acid also includes detecting the nucleic acid. For example, in determining the presence or absence of a mutation in JAK2, the JAK2 nucleic acid is also detected. Methods of determining the presence or absence of one or more mutations may include a variety of methods known in the art including one or more of reverse transcribing JAK2 RNA to cDNA, amplifying JAK2 nucleic acid, hybridizing a probe or a primer to JAK2 nucleic acid, and sequencing JAK2 nucleic acid.

The term “oligonucleotide” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group in this position. Oligonucleotides also may include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including chemical synthesis, DNA replication, reverse transcription, PCR, or a combination thereof. The oligonucleotide may be modified. For example, the oligonucleotide may be labeled with an agent that produces a detectable signal (e.g., a fluorophore).

“Primer” refers to an oligonucleotide that is capable of acting as a point of initiation of synthesis when placed under conditions in which primer extension is initiated (e.g., primer extension associated with an application such as PCR). An oligonucleotide “primer” may occur naturally, as in a purified restriction digest or may be produced synthetically.

A “probe” refers to an oligonucleotide that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid.

A “target nucleic acid” refers to a nucleic acid molecule containing a sequence that has at least partial complementarity with a probe oligonucleotide and/or a primer oligonucleotide. A probe may specifically hybridize to a target nucleic acid.

As used herein, the term “activation domain” in reference to JAK2 refers generally to a domain involved in cell activation. An example of an activation domain is a kinase or pseudokinase domain.

As used herein, the term “pseudokinase domain” refers to a portion of a polypeptide or nucleic acid that encodes a portion of the polypeptide, where the portion shows homology to a functional kinase but possesses no catalytic activity. A pseudokinase domain may also be referred to as a “kinase-like domain.” An example of a pseudokinase domain is the JAK2 psuedokinase domain, also termed the JH2 domain, represented within SEQ ID NO: 2, FIG. 2.

The term “kinase domain” refers to a portion of a polypeptide or nucleic acid that encodes a portion of the polypeptide, where the portion is required for kinase activity of the polypeptide (e.g., tyrosine kinase activity).

In some methods of the invention, mutations may “affect JAK2 kinase activity.” The affected JAK2 kinase activity may include kinase activity that increases, decreases, becomes constitutive, stops completely or affects greater, fewer or different targets. A mutation that affects kinase activity may be present in a kinase domain or in a domain associated with a kinase domain such as the JAK2 pseudokinase domain.

The term “inhibitor” as used herein refers to any substance, molecule, or drug that when properly administered, decreases, downwardly modulates, or prohibits a reaction or an activity.

The term “amplification” or “amplifying” refers to the production of additional copies of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, and nucleoside triphosphates, and optionally at least one labeled probe and/or optionally at least one agent for determining the melting temperature of an amplified target nucleic acid (e.g., a fluorescent intercalating agent that exhibits a change in fluorescence in the presence of double-stranded nucleic acid).

As used herein, the term “including” has the same meaning as the term comprising.

As used herein, the term “about” means in quantitative terms, plus or minus 10%.

As used herein, the term “treatment,” “treating,” or “treat” refers to care by procedures or application that are intended to relieve illness or injury. Although it is preferred that treating a condition or disease such as a myeloproliferative disease will result in an improvement of the condition, the term treating as used herein does not indicate, imply, or require that the procedures or applications are at all successful in ameliorating symptoms associated with any particular condition. Treating a patient may result in adverse side effects or even a worsening of the condition which the treatment was intended to improve.

As used herein the terms “diagnose” or “diagnosis” or “diagnosing” refer to distinguishing or identifying a disease, syndrome or condition or distinguishing or identifying a person having a particular disease, syndrome or condition.

As used herein, the term “assay” or “assaying” means qualitative or quantitative analysis or testing.

As used herein the term “ratio” refers to the relation in degree or number between two similar things. For example, the relative amount of mutant to wild-type nucleic acid in a sample may be referred to as a ratio of wild-type to mutant nucleic acid.

As used herein the term “sequencing” as in determining the sequence of a polynucleotide refers to methods that determine the base identity at multiple base positions or determine the base identity at a single position.

### BRIEF DESCRIPTION OF THE FIGURES

FIG. 1 is a nucleic acid sequence of JAK2.

FIG. 2 is a nucleic acid sequence of the JAK2 pseudokinase domain region.

FIG. 3 is an amino acid sequence of JAK2.

FIG. 4 is an amino acid sequence of the JAK2 pseudokinase domain region.

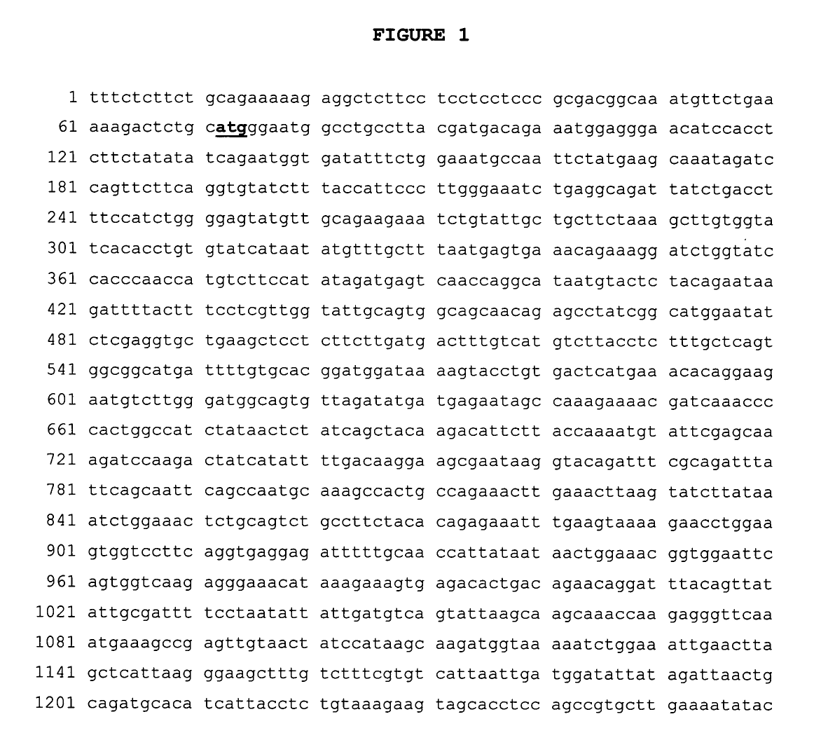

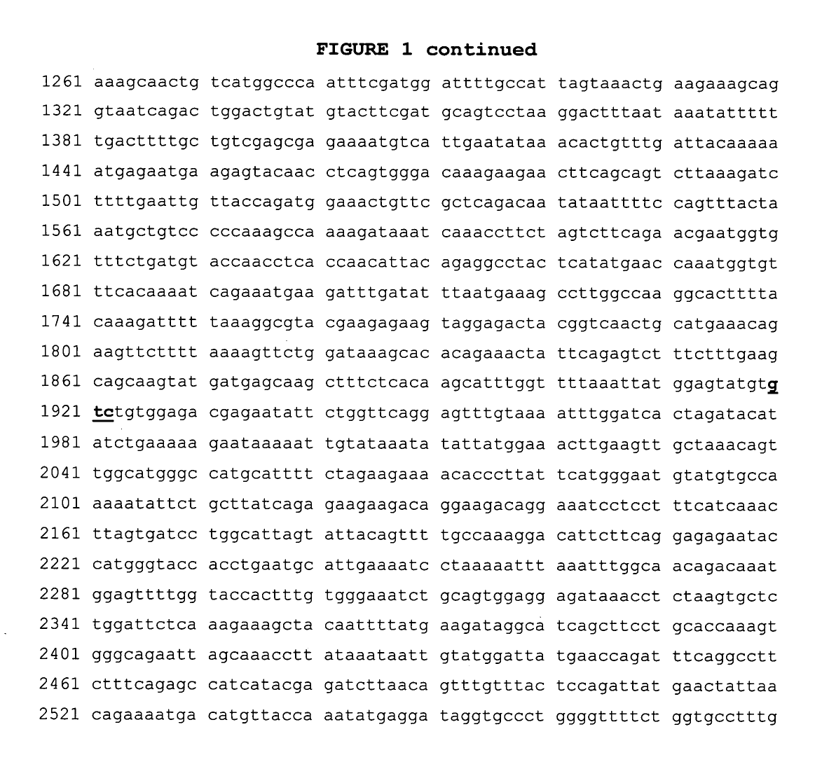

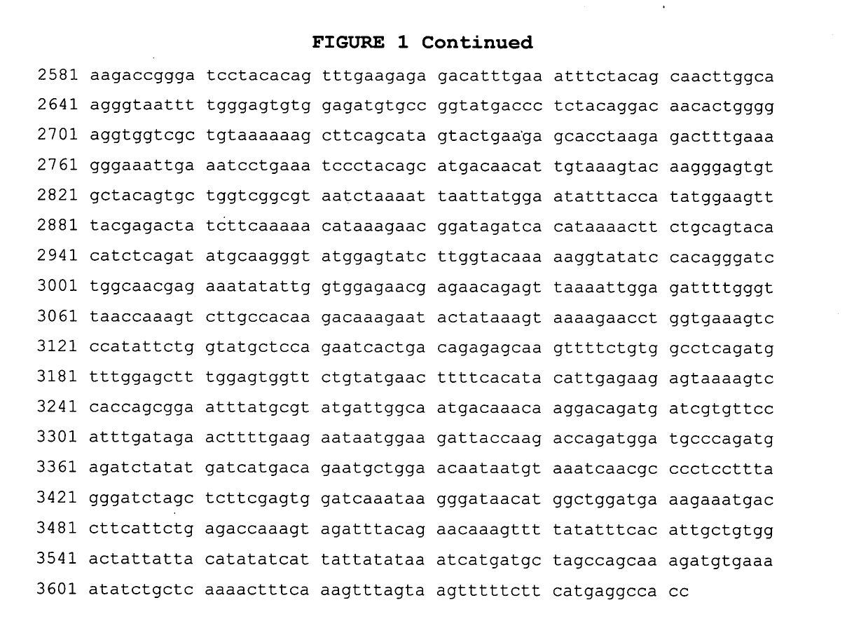

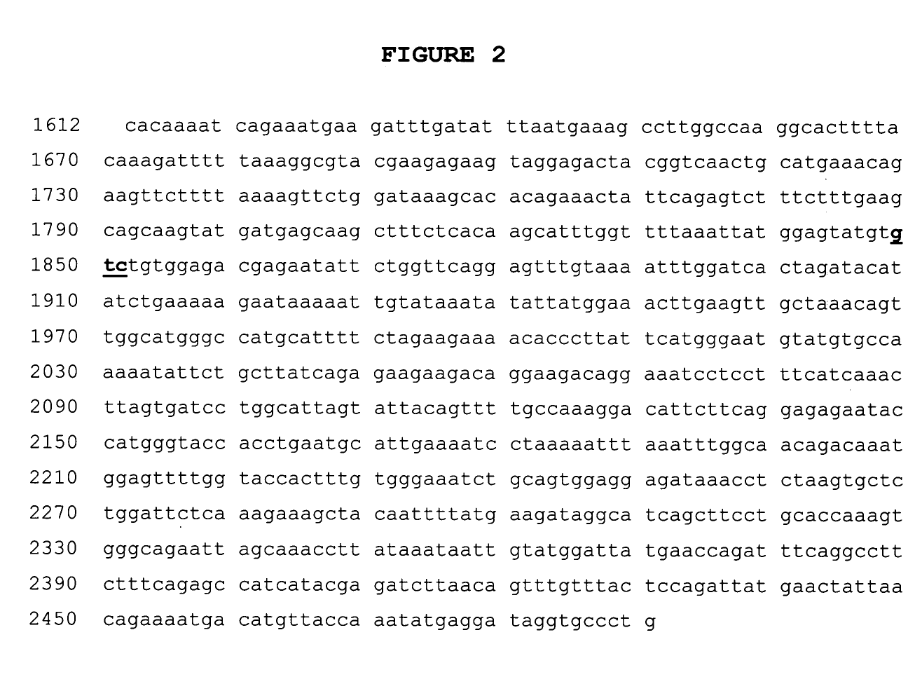

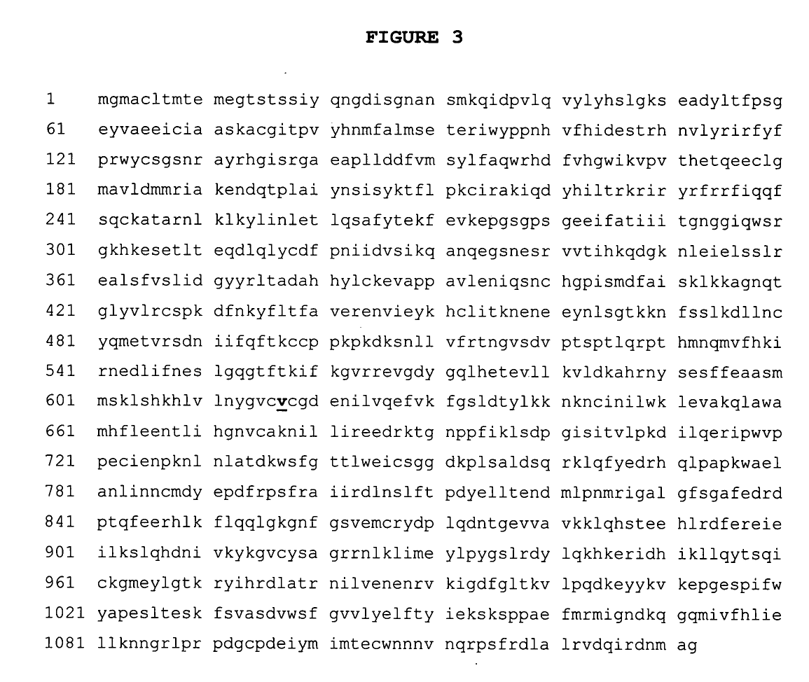

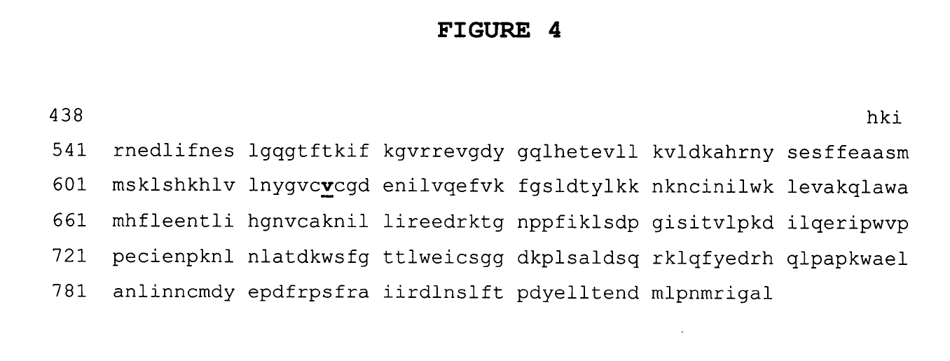

### DETAILED DESCRIPTION OF THE INVENTION

The specific gene and concomitant-mutation or mutations responsible for many myeloproliferative diseases (“MPDs”) is not known. However, a mutation in the Janus kinase 2 (JAK2) gene, a cytoplasmic, nonreceptor tyrosine kinase, has been identified in a number of MPDs. For example, this mutation has been reported in up to 97% of patients with PV, and in greater than 40% of patients with either ET or IMF. See e.g., Baxter et al., Lancet 365:1054-1060 (2005); James et al., Nature 438:1144-1148 (2005); Zhao, et al., J. Biol. Chem. 280(24):22788-22792 (2005); Levine et al., Cancer Cell, 7:387-397 (2005); Kralovics, et al., New Eng. J. Med. 352(17):1779-1790 (2005).

The Janus kinases are a family of tyrosine kinases that play a role in cytokine signaling. For example, JAK2 kinase acts as an intermediary between membrane-bound cytokine receptors such as the erythropoietin receptor (EpoR), and down-stream members of the signal transduction pathway such as STAT5 (Signal Transducers and Activators of Transcription protein 5). See, e.g, Tefferi and Gilliland, Mayo Clin. Proc. 80:947-958 (2005); Giordanetto and Kroemer, Protein Engineering, 15(9):727-737 (2002). JAK2 is activated when cytokine receptor/ligand complexes phosphorylate the associated JAK2 kinase. Id. JAK2 can then phosphorylate and activate its substrate molecule, for example STAT5, which enters the nucleus and interacts with other regulatory proteins to affect transcription. Id. In the JAK2 mutant, a valine (codon “GTC”) is replaced by a phenylalanine (codon “TTC”) at amino acid position 617 (the “V617F mutant”). Baxter et al., Lancet 365:1054-1060 (2005). Amino acid 617 is located in exon 12 which includes a pseudokinase, auto-inhibitory (or negative regulatory) domain termed JH2 (Jak Homology 2 domain). Id.; James et al., Nature 438:1144-1148 (2005). Though this domain has no kinase activity, it is thought to interact with the JH1 (Jak Homology 1) domain, which does have kinase activity. Baxter et al., Lancet 365:1054-1060 (2005). Appropriate contact between the two domains in the wild-type protein allows proper kinase activity and regulation; however, the V617F mutation causes improper contact between the two domains, resulting in constitutive kinase activity in the mutant JAK2 protein. Id.

The presence of the JAK2 V617F mutation can be as an indicator of disease. Additionally, using the methods of the invention, we show that the zygosity status of the JAK2 V617F mutation is prognostic of disease progression and patient longevity for some patient populations (described below). Also, the ratio of the mutant to wild-type nucleic acid in a patient sample may be used to monitor facts such as disease progression and treatment efficacy.

The zygosity status and the ratio of wild-type to mutant nucleic acid in a sample may be determined by methods known in the art including sequence-specific, quantitative detection methods. Other methods may involve determining the area under the curves of the sequencing peaks from standard sequencing electropherograms, such as those created using ABI Sequencing Systems, (Applied Biosystems, Foster City Calif.). For example, the presence of only a single peak such as a “G” on an electropherogram in a position representative of a particular nucleotide is an indication that the nucleic acids in the sample contain only one nucleotide at that position, the “G.” The sample may then be categorized as homozygous because only one allele is detected. The presence of two peaks, for example, a “G” peak and a “T” peak in the same position on the electropherogram indicates that the sample contains two species of nucleic acids; one species carries the “G” at the nucleotide position in question, the other carries the “T” at the nucleotide position in question. The sample may then be categorized as heterozygous because more than one allele is detected.

The sizes of the two peaks may be determined (e.g, by determining the area under each curve), and a ratio of the two different nucleic acid species may be calculated. A ratio of wild-type to mutant nucleic acid may be used to monitor disease progression, determine treatment or to make a diagnosis. For example, the number of cancerous cells carrying the JAK2 V617F mutation may change during the course of an MPD. If a base line ratio is established early in the disease, a later determined higher ratio of mutant nucleic acid relative to wild-type nucleic acid may be an indication that the disease is becoming worse or a treatment is ineffective; the number of cells carrying the mutation may be increasing in the patient. A lower ratio of mutant relative to wild-type nucleic acid may be an indication that a treatment is working or that the disease is not progressing; the number of cells carrying the mutation may be decreasing in the patient.

Samples

The methods of the invention may be performed with a variety of patient sample types. In preferred embodiments, acellular bodily fluids, such as plasma or serum are used. It has been found that detection of JAK2 mutations from plasma is at least as sensitive if not more so, than detection of JAK2 mutations from blood or bone marrow cells. In a simultaneous analysis of plasma and peripheral blood cells from 30 patients with myeloproliferative disease, it was found that all samples that tested positive for the V617F mutation in cells also tested positive for this mutation in plasma. However, hemizygosity/homozygosity at the mutant locus was more evident in sequences from plasma, whereas cells of the same patient displayed both mutant and wild-type sequences. Heterozygous plasma samples showed equally intense wild-type (“G”) and mutant (“T”) peaks (in sequencing reactions), whereas the mutant “T” peak was less conspicuous in the cell sample. This suggests that the essentially cell-free plasma is enriched with tumor-specific nucleic acid, and that the cell samples, containing populations of both malignant and non-malignant cells (e.g., lymphocytes carrying wild-type alleles of JAK2), give little or poor information regarding homozygous and hemizygous cell populations. Thus, perhaps due to the clonal nature of the JAK2 V617F mutation, detection of mutations from plasma samples appears superior to detection methods using cellular samples.

Diagnosis

One or more of the following determinations may be used to diagnose a patient: determining the presence or absence of a JAK2 V617F mutation, determining the zygosity status of the sample, and determining the ratio of mutant to wild-type JAK2 nucleic acid in the sample. For example, patients found to carry the JAK2 V617F mutation by the methods of the invention may be recommended for further testing to verify an MPD diagnosis, or detection of the mutation may be used to finally confirm a preliminary diagnosis of MPD (e.g., if a patient is symptomatic for an MPD and also tests positive for the V617F mutation, the patient may be finally diagnosed with an MPD such as PV). Similarly, methods of the invention may be used to diagnose patients who are asymptomatic for MPD, for example patients who are in the very early stages of an MPD. JAK2 mutations may also be detected in MPD patients who are undergoing treatment; if the ratio of mutant to wild-type JAK2 nucleic acid or the zygosity status of the sample changes during treatment, a different diagnosis may be made.

Treatment

One or more of the following determinations may be used to treat a patient: determining the presence or absence of a JAK2 V617F mutation, determining the zygosity status of the sample, and determining the ratio of mutant to wild-type JAK2 nucleic acid in a sample. A physician or treatment specialist may administer, forego or alter a treatment or treatment regime based on one or more of the determinations. For example, a doctor might administer a specific kinase inhibitor directed to the JAK2 mutant protein that could stop or attenuate the constitutive kinase activity. This treatment would be administered if the mutation was present in the patient sample. Conversely, a doctor may forego such a treatment if the mutation is not present.

Additionally, one or more of the determinations could aid in patient prognosis and quality of life decisions. For example, decisions about whether to continue—or for how long to continue—a painful, debilitating treatment such as chemotherapy could be made.

Further, the number of cancerous cells carrying the mutation may change during the course of an MPD and monitoring the ratio, the zygosity status or the presence or absence of a JAK2 mutation could be an indication of disease status or treatment efficacy. For example, treatment may reduce the number of mutant cancerous cells, or the disease could become worse with time, and the number of diseased cells may increase. Accordingly, a treatment may be administered, changed, or foregone based on changes in zygosity status or the ratio of wild-type to mutant nucleic acid in a patient sample.

Methods

Numerous methods to collect and process patient samples, isolate or purify nucleic acid, and determine the presence or absence of a JAK2 V617F mutation are known. One exemplary embodiment, from sample preparation to detection, is described below. However, it is understood that one skilled in the art could properly select, combine and utilize the variety of individual method steps described.

Total nucleic acid may be extracted from patient plasma or peripheral blood cells using NucliSens extraction kit (Biomerieux, Marcy I'Etoile, France). In other methods, mRNA may be extracted from patient blood/bone marrow samples using MagNA Pure LC mRNA HS kit and Mag NA Pure LC Instrument (Roche Diagnostics Corporation, Roche Applied Science, Indianapolis, Ind.). Next, an RT-PCR reaction may be performed using either the total nucleic acid preparation or the RNA preparation to specifically amplify a portion of the patient RNA. An exemplary one-step RT-PCR system is the Superscript III System (Invitrogen, Carlsbad, Calif.). Other methods and systems for RT-PCR reactions are well known in the art and are commercially available. A primer pair is designed to encompass a region of interest, for example, the V617F mutation in JAK2 nucleic acid, to yield a PCR product. By way of example, but not by way of limitation, a primer pair for JAK2 may be 5′-GAC TAC GGT CAA CTG CAT GAA A-3′, and 5′-CCA TGC CAA CTG TTT AGC AA-3′ (SEQ ID NOs: 5 and 6). The resulting RT-PCR product is 273 nucleotides long. The RT-PCR product may then be purified, for example by gel purification, and the resulting purified product may be sequenced. Nucleic acid sequencing methods are known in the art; an exemplary sequencing method includes the ABI Prism BIgDye Terminator v3.1 Cycle Sequencing Kit (Applied Biosystems, Foster City, Calif.). The sequencing data may then be analyzed for the presence or absence of one or more mutations in JAK2 nucleic acid. The sequencing data may also be analyzed to determine the proportion of wild-type to mutant nucleic acid present in the sample.

Plasma or Serum Preparation Methods

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70 degrees centigrade until thawed and used. “Fresh” plasma or serum should be refrigerated or maintained on ice until used, with nucleic acid (e.g., RNA, DNA or total nucleic acid) extraction being performed as soon as possible. Exemplary methods are described below.

Blood can be drawn by standard methods into a collection tube, preferably siliconized glass, either without anticoagulant for preparation of serum, or with EDTA, sodium citrate, heparin, or similar anticoagulants for preparation of plasma. The preferred method if preparing plasma or serum for storage, although not an absolute requirement, is that plasma or serum be first fractionated from whole blood prior to being frozen. This reduces the burden of extraneous intracellular RNA released from lysis of frozen and thawed cells which might reduce the sensitivity of the amplification assay or interfere with the amplification assay through release of inhibitors to PCR such as porphyrins and hematin. “Fresh” plasma or serum may be fractionated from whole blood by centrifugation, using preferably gentle centrifugation at 300-800 times gravity for five to ten minutes, or fractionated by other standard methods. High centrifugation rates capable of fractionating out apoptotic bodies should be avoided. Since heparin may interfere with RT-PCR, use of heparinized blood may require pretreatment with heparinase, followed by removal of calcium prior to reverse transcription. Imai, H., et al., J. Virol. Methods 36:181-184, (1992). Thus, EDTA is the preferred anticoagulant for blood specimens in which PCR amplification is planned.

Nucleic Acid Extraction from Cells and Acellular Bodily Fluids

Numerous methods are known in the art for isolating total nucleic acid, DNA and RNA from blood, serum, plasma and bone marrow or other hematopoietic tissues. In fact, numerous published protocols, as well as commercial kits and systems are available. By way of example but not by way of limitation, examples of such kits, systems and published protocols are described below. Commercially available kits include Qiagen products such as the QiaAmp DNA Blood MiniKit (Cat.# 51104, Qiagen, Valencia, Calif.), the QiaAmp RNA Blood MiniKit (Cat.# 52304, Qiagen, Valencia, Calif.); Promega products such as the Wizard Genomic DNA Kit (Cat.# A1620, Promega Corp. Madison, Wis.), Wizard SV Genomic DNA Kit (Cat.# A2360, Promega Corp. Madison, Wis.), the SV Total RNA Kit (Cat.# X3100, Promega Corp. Madison, Wis.), PolyATract System (Cat.# Z5420, Promega Corp. Madison, Wis.), or the PurYield RNA System (Cat.# Z3740, Promega Corp. Madison, Wis.).

Other methods include the following. Whole blood or bone marrow samples can be drawn into ACD or EDTA anticoagulant, or frozen plasma samples may be used. Blood may stored at room temperature or refrigerated. Plasma may be stored frozen. Total Nucleic acid may be extracted from plasma samples using bioMerieux NucliSens miniMAG or Easy MAG Nucliec Acid Purification System (bioMerieux SA, Marcy I'Etoile, France) or equivalent. Alternatively, mRNA may be extracted from blood/bone marrow samples using MagNA Pure LC mRNA HS Kit and MagNA Pure LC Instrument (Roche Diagnostics, Roche Molecular Systems, Inc., Alameda, Calif.) or equivalent.

Another exemplary method, published by Kantarjian, H., et al., Clin. Cancer Res. 9:160-6 (2003), is as follows. Patient white blood cells are isolated from 10-20 milliliters of peripheral blood or 1-3 milliliters of bone marrow by treatment with two cycles of ammonium chloride buffer. Total RNA is extracted with Trizol (Life Technologies, Inc.) from about 1×10⁵ to 1×10⁷ white blood cells or bone marrow mononuclear cells. After measuring the concentration of RNA by, for example, a spectrophotometric method, (Beckman DU640B; Palo Alto, Calif.) RNA is transcribed into cDNA using the procedure described previously by Cross, N. C., et al., Blood 82: 1929-36, (1993), or by methods known in the art, some of which are described below.

Extraction of RNA from Plasma or Serum

Numerous methods are known in the art for extracting RNA from body fluids such as plasma or serum and any suitable method may be used. Previously described methods, kits or systems for extraction of mammalian RNA or viral RNA may be adapted, either as published or modified for the extraction of tumor-derived or associated RNA from plasma or serum. For example, Roche MagNA Pure RNA extraction system and methods (Roche Diagnostics, Roche Molecular Systems, Inc., Alameda, Calif.), may be used. Or, methods described in U.S. Pat. No. 6,916,634 may also be employed. Additional examples of RNA extraction are described below.

The volume of plasma or serum used in the extraction may be varied dependent upon clinical intent, but volumes of 100 microliters to one milliliter of plasma or serum are sufficient, with the larger volumes often indicated in settings of minimal or premalignant disease.

Glass beads, Silica particles or Diatom Extraction

RNA may be extracted from plasma or serum using silica particles, glass beads, or diatoms, as in the method or adaptations of Boom, R., et al., J. Clin. Micro. 28:495-503, (1990). Application of the method adapted by Cheung, R. C., et al., J Clin Micro. 32:2593-2597, (1994), is described.

Size fractionated silica particles are prepared by suspending 60 grams of silicon dioxide (SiO2, Sigma Chemical Co., St. Louis, Mo.) in 500 milliliters of demineralized sterile double-distilled water. The suspension is then settled for 24 hours at room temperature. Four-hundred thirty (430) milliliters of supernatant is removed by suction and the particles are resuspended in demineralized, sterile double-distilled water added to equal a volume of 500 milliliters. After an additional 5 hours of settlement, 440 milliliters of the supernatant is removed by suction, and 600 microliters of HCl (32% wt/vol) is added to adjust the suspension to a pH2. The suspension is aliquotted and stored in the dark.

Lysis buffer is prepared by dissolving 120 grams of guinidine thiocyanate (GuSCN, Fluka Chemical, Buchs, Switzerland) into 100 milliliters of 0.1 M Tris hydrochloride (Tris-HCl) (pH 6.4), and 22 milliliters of 0.2 M EDTA, adjusted to pH 8.0 with NaOH, and 2.6 grams of Triton X-100 (Packard Instrument Co., Downers Grove, Ill.). The solution is then homogenized.

Washing buffer is prepared by dissolving 120 grams of guinidine thiocyanate (GuSCN) into 100 milliliters of 0.1 M Tris-HCl (pH 6.4).

One hundred microliters to two hundred fifty microliters (with greater amounts required in settings of minimal disease) of plasma or serum are mixed with 40 microliters of silica suspension prepared as above, and with 900 microliters of lysis buffer, prepared as above, using an Eppendorf 5432 mixer over 10 minutes at room temperature. The mixture is then centrifuged at 12,000×g for one minute and the supernatant aspirated and discarded. The silica-RNA pellet is then washed twice with 450 microliters of washing buffer, prepared as above. The pellet is then washed twice with one milliliter of 70% (vol/vol) ethanol. The pellet is then given a final wash with one milliliter of acetone and dried on a heat block at 56 degrees centigrade for ten minutes. The pellet is resuspended in 20 to 50 microliters of diethyl procarbonate-treated water at 56 degrees centigrade for ten minutes to elute the RNA. The sample can alternatively be eluted for ten minutes at 56 degrees centigrade with a TE buffer consisting of 10 millimolar Tris-ris-HCl-one millimolar EDTA (pH 8.0) with an RNase inhibitor (RNAsin, 0.5 U/microliter, Promega), with or without Proteinase K (100 ng/ml) as described by Boom, R., et al., J. Clin. Micro. 29:1804-1811, (1991). Following elution, the sample is then centrifuged at 12,000×g for three minutes, and the RNA containing supernatant recovered.

Acid Guanidinium Thiocyanate-Phenol-Chloroform Extraction

As an alternative method, RNA may be extracted from plasma or serum using the Acid Guanidinium Thiocyanate-Phenol-chloroform extraction method described by Chomczynski, P. and Sacchi, N., Analytical Biochemistry 162:156-159, (1987), as follows.

The denaturing solution consists of 4 M guanidinium thiocyanate, 25 millimolar sodium citrate, pH 7.0, 0.5% sarcosyl, and 0.1 M 2-mercaptoethanol. The denaturing solution is prepared as follows: A stock solution is prepared by dissolving 250 grams of guanidinium thiocyanate (GuSCN, Fluka Chemical) with 293 milliliters of demineralized sterile double-distilled water, 17.6 milliliters of 0.75 M sodium citrate, pH 7.0, and 26.4 milliliters of 10% sarcosyl at 65 degrees centigrade. The denaturing solution is prepared by adding 0.36 milliliters 2-mercaptoethanol/50 milliliters of stock solution.

One hundred microliters to one milliliter of body fluid is mixed with one milliliter of denaturing solution. Sequentially, 0.1 milliliter of 2 M sodium acetate, pH 4.0, 1 milliliter of phenol, and 0.2 milliliter of chloroform-isoamyl alcohol (49:1) are added, with mixing after addition of each reagent. The resultant mixture is shaken vigorously for 10 seconds, cooled on ice for 15 minutes, and then centrifuged at 10,000×g for 20 minutes at 4 degrees centigrade. The aqueous phase is then transferred to a clean tube and mixed with 1 milliliter of isopropanol. The mixture is then cooled at −20 degrees centigrade for 1-2 hours to precipitate RNA. After centrifugation at 10,000×g for 20 minutes the resulting RNA pellet is dissolved in 0.3 milliliter of denaturing solution, and then reprecipitated with 1 volume isopropanol at −20 degrees centigrade for one hour. Following another centrifugation at 10,000×g for ten minutes at 4 degrees centigrade, 75% ethanol is added to resuspend the RNA pellet, which is then sedimented and vacuum dried, and then dissolved in 5-25 microliters of 0.5% SDS at 65 degrees centigrade for ten minutes. The RNA extract is now ready for further analysis.

As an alternative method, RNA may be extracted from plasma or serum using variations of the acid guanidinium thiocyanate-phenol-chloroform extraction method. For example, in the preferred embodiment RNA is extracted from plasma or serum using TRI reagent, a monophase guanidine-thiocyanate-phenol solution, as described by Chomczynski, P., Biotech 15:532-537, (1993). One hundred microliters to one milliliter of plasma or serum is processed using one milliliter of TRI Reagent™ (TRI Reagent, Sigma Trisolv™, BioTecx Laboratories, Houston, Tex., TRIzol™, GIBCO BRL/Life Technologies, Gaithersburg, Md.) according to manufacturer's directions. Minor adaptations may be applied as currently practiced within the art. Thus, from one hundred microliters to one milliliter of plasma or serum is mixed with one milliliter of TRI Reagent. Then 0.2 milliliter of chloroform is mixed for 15 seconds, and the mixture allowed to stand for 3 minutes at room temperature. The mixture is then centrifuged at 4 degrees centigrade for 15 minutes at 12,000×g. The upper aqueous phase is removed to which 0.5 milliliter of isopropanol is mixed, and then left at room temperature for five minutes, followed by centrifugation at 4 degrees centigrade for ten minutes at 12,000×g. The RNA pellet is then washed with one milliliter of 75% ethanol by centrifuging at 12,000×g for 5 minutes. The pellet is air dried and resuspended in 11.2 microliters of RNAse free water.

Contamination by polysaccharides and proteoglycans, which may be present in extracellular proteolipid-RNA complexes, may be reduced by modification of the precipitation step of the TRI Reagent™ procedure, as described by Chomczynski, P. and Mackey, K., BioTechniques 19:942-945, (1995), as follows.

One hundred microliters to one milliliter of body fluid is mixed with TRI Reagent™ as per manufacturer's directions, being subjected to phase separation using either chloroform or bromo-chloropropane, as described by Chomczynski, P. and Mackey, K., Analytical Biochemistry 225:163-164, (1995), and centrifugation at 10,000×g for 15 minutes. The aqueous phase is removed and then mixed with 0.25 milliliters of isopropanol followed with 0.25 milliliters of a high-salt precipitation solution (1.2 M NaCl and 0.8 M sodium citrate). The mixture is centrifuged at 10,000×g for 5 minutes and washed with one milliliter of 75% ethanol. The RNA pellet is then vacuum dried and then dissolved in 5-25 microliters of 0.5% SDS at 65 degrees centigrade for ten minutes.

Alternative Methods

Alternative methods may be used to extract RNA from body fluids including but not limited to centrifugation through a cesium chloride gradient, including the method as described by Chirgwin, J. M., et al., Biochemistry 18:5294-5299, (1979), and co-precipitation of extracellular RNA from plasma or serum with gelatin, such as by adaptations of the method of Fournie, G. J., et al., Analytical Biochemistry 158:250-256, (1986), to RNA extraction.

Circulating extracellular deoxyribonucleic acid (DNA), including tumor-derived or associated extracellular DNA, is also present in plasma and serum. See Stroun, M., et al., Oncology 46:318-322, (1989). Since this DNA will additionally be extracted to varying degrees during the RNA extraction methods described above, it may be desirable or necessary (depending upon clinical objectives) to further purify the RNA extract and remove trace DNA prior to proceeding to further RNA analysis. This may be accomplished using DNase, for example by the method as described by Rashtchian, A., PCR Methods Applic. 4:S83-S91, (1994), as follows.

For one microgram of RNA, in a 0.5 milliliter centrifuge tube placed on ice, add one microliter of 10×DNase I reaction buffer (200 micromolar Tris-HCl (pH 8.4), 500 micromolar KCl, 25 micromolar MgCl₂, one micromolar per milliliter bovine serum albumin). Add to this one unit DNase I (GIBCO/BRL catalog #18068-015). Then bring the volume to ten microliter with DEPC-treated distilled water, and follow by incubating at room temperature for 15 minutes. The DNase I is then inactivated by the addition of 20 millimolar EDTA to the mixture, and heating for 10 minutes at 65 degrees centigrade.

Alternatively, primers for further RNA analysis may be constructed which favor amplification of the RNA products, but not of contaminating DNA, such as by using primers which span the splice junctions in RNA, or primers which span an intron. Alternative methods of amplifying RNA but not the contaminating DNA include the methods as described by Moore, R. E., et al., Nucleic Acids Res. 18:1921, (1991), and methods as described by Buchman, G. W., et al., PCR Methods Applic. 3:28-31, (1993), which employs a dU-containing oligonucleotide as an adaptor primer.

Nucleic Acid Amplification and Mutation Detection

Nucleic acid extracted from tissues, cells, plasma or serum can be amplified using nucleic acid amplification techniques well know in the art. Many of these amplification methods can also be used to detect the presence of mutations simply by designing oligonucleotide primers or probes to interact with or hybridize to a particular target sequence in a specific manner. By way of example, but not by way of limitation these techniques can include the polymerase chain reaction (PCR) reverse transcriptase polymerase chain reaction (RT-PCR), nested PCR, ligase chain reaction. See Abravaya, K., et al., Nucleic Acids Research 23:675-682, (1995), branched DNA signal amplification, Urdea, M. S., et al., AIDS 7 (suppl 2):S11-S 14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods 35:273-286, (1991), Invader Technology, or other sequence replication assays or signal amplification assays.

Reverse Transcription of RNA to cDNA

Some methods employ reverse transcription of RNA to cDNA. As noted, the method of reverse transcription and amplification may be performed by previously published or recommended procedures, which referenced publications are incorporated herein by reference in their entirety. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus Thermophilus. For example, one method, but not the only method, which may be used to convert RNA extracted from plasma or serum to cDNA is the protocol adapted from the Superscript II Preamplification system (Life Technologies, GIBCO BRL, Gaithersburg, Md.; catalog no. 18089-011), as described by Rashtchian, A., PCR Methods Applic. 4:S83-S91, (1994), adapted as follows.

One (1) to five (5) micrograms of RNA extracted from plasma or serum in 13 microliters of DEPC-treated water is added to a clean microcentrifuge tube. Then one microliter of either oligo (dT) (0.5 milligram/milliliter) or random hexamer solution (50 ng/microliter) is added and mixed gently. The mixture is then heated to 70 degrees centigrade for 10 minutes and then incubated on ice for one minute. Then, it is centrifuged briefly followed by the addition of 2 microliters of Oxsynthesis buffer (200 mM Tris-HCl, pH 8.4, 500 mM KCl, 25 mm magnesium chloride, one milligram/milliliter of BSA), one microliter of 10 mM each of dNTP mix, 2 microliters of 0.1 M DTT, one microliter of SuperScript II RT (200 U/microliter) (Life Technologies, GIBCO BRL, Gaithersburg, Md.). After gentle mixing, the reaction is collected by brief centrifugation, and incubated at room temperature for ten minutes. The tube is then transferred to a 42 degrees centigrade water bath or heat block and incubated for 50 minutes. The reaction is then terminated by incubating the tube at 70 degrees centigrade for 15 minutes, and then placing it on ice. The reaction is collected by brief centrifugation, and one microliter of RNase H (2 units) is added followed by incubation at 37 degrees centigrade for 20 minutes before proceeding to nucleic acid amplification.

Nucleic Acid Amplification

To the cDNA mixture add the following: 8 microliters of 10× synthesis buffer (200 mM Tris-HCl, pH 8.4, 500 mM KCl, 25 mM magnesium chloride, 1 mg/ml of BSA), 68 microliters sterile double-distilled water, one microliter amplification primer 1 (10 micromolar), one microliter amplification primer 2 (10 micromolar), one microliter Taq DNA polymerase (2-5 U/microliter). Mix gently and overlay the reaction mixture with mineral oil. The mixture is heated to 94 degrees centigrade for 5 minutes to denature remaining RNA/cDNA hybrids. PCR amplification is then performed in an automated thermal-cycler for 15-50 cycles, at 94 degrees centigrade for one minute, 55 degrees centigrade for 30 to 90 seconds, and 72 degrees centigrade for 2 minutes.

Cycling parameters and magnesium concentration may vary depending upon the specific sequence to be amplified, however, optimization procedures and methods are also well known in the art.

Also, primers may contain appropriate restriction sites, and restriction digestion may be performed on the amplified product to allow further discrimination between mutant and wild-type sequences.

Alternative Methods

Alternative methods of nucleic acid amplification which may be used include variations of RT-PCR, including quantitative RT-PCR, for example as adapted to the method described by Wang, A. M. et al., PNAS USA 86:9717-9721, (1989), or by Karet, F. E., et al., Analytical Biochemistry 220:384-390, (1994).

An alternative method of nucleic acid amplification or mutation detection which may be used is ligase chain reaction (LCR), as described by Wiedmann, et al., PCR Methods Appl. 3:551-564, (1994). In the ligase chain reaction, RNA extracted from plasma or serum is reverse transcribed to cDNA. LCR is a a technique to detect single base mutations. A primer is synthesized in two fragments and annealed to the template with possible mutation at the boundary of the two primer fragments. Ligase will ligate the two fragments if they match exactly to the template sequence. Subsequent PCR reactions will amplify only if the primer is ligated. Restriction sites can also be utilized to discriminate between mutant and wild-type sequences.

An alternative method of amplification or mutation detection is allele specific PCR (ASPCR). ASPCR which utilizes matching or mismatching between the template and the 3′ end base of a primer well known in the art. See e.g., U.S. Pat. No. 5,639,611.

Another alternative method of amplification or mutation detection which may be used is branched DNA signal amplification, for example as adapted to the method described by Urdea, M. S., et al., AIDS 7 (suppl 2):S11-S 14, (1993), with modification from the reference as follows: RNA is extracted from plasma or serum and then added directly to microwells. The method for detection of tumor-related or tumor-associated RNA then proceeds as referenced in Urdea, et al, Id., with target probes specific for the tumor-related or tumor-associated RNA or cDNA of interest, and with chemiluminescent light emission proportional to the amount of tumor-associated RNA in the plasma or serum specimen. The specifics of the referenced method are described further by Urdea, M. S., et al., Nucleic Acids Research Symposium Series 24:197-200, (1991), with this reference incorporated herein in its entirety.

An alternative method of either amplification or mutation detection which may be used is isothermal nucleic acid sequence based amplification (NASBA), for example as adapted to the method described by Kievits, T., et al., J Virological Methods 35:273-286, (1991), or by Vandamme, A. M., et al., J. Virological Methods 52:121-132, (1995).

Alternative methods of either qualitative or quantitative amplification of nucleic acids which may be used, but are not limited to, Q-beta replication, other self-sustained sequence replication assays, transcription-based amplification assays, and amplifiable RNA reporters, boomerang DNA amplification, strand displacement activation, and cycling probe technology.

Another method of mutation detection is nucleic acid sequencing. Sequencing can be performed using any number of methods, kits or systems known in the art. One example is using dye terminator chemistry and an ABI sequencer (Applied Biosystems, Foster City, Calif.). Sequencing also may involve single base determination methods such as single nucleotide primer extension (“SNapShot” sequencing method) or allele or mutation specific PCR.

The versatility of the invention is illustrated by the following Examples which illustrate preferred embodiments of the invention and are not limiting of the claims or specification in any way.

### EXAMPLES

1. Determining the Sensitivity of JAK2 Mutation Detection from Plasma

The sensitivity of detecting JAK2 nucleic acid from plasma was determined as follows. A HEL cell line (92.1.7, obtained from the American Type Culture Collection, Manassas, Va.), carrying only the JAK2 V617F mutation (no wild-type allele), was maintained in RPMI 1640 with 10% fetal calf serum. A lysate was prepared and combined with plasma from normal (JAK2 wild-type) individuals at various concentrations.

Total RNA was extracted from the mixtures using the NucliSense Extraction Kit (bioMerieux Inc., Durham, N.C.) as recommended by the manufacturer. A PCR primer pair was designed to amplify across the region of the JAK2 gene coding for amino acid 671. The primer sequences used for PCR and sequencing were as follows: JAK2-F (5′-GAC TAC GGT CAA CTG CAT GAA A-3′) SEQ ID NO: 5, and JAK2-R (5′-CCA TGC CAA CTG TTT AGC AA-3′) SEQ ID NO: 6. One-step RT-PCR was performed in a 25 μL reaction volume using SuperScript III one-step RT-PCR system with Platinum Taq (Invitrogen, Carlsbad, Calif.). Concentrations used for RT-PCR were: 1× reaction buffer, 400 nM each of the forward and reverse JAK2 primers, 1 unit of SupersScript III and 5 μL of the RNA template. The thermocycler conditions were: 30 minutes at 55° C. for reverse transcription, followed by 2 minutes at 94° C. and 40 cycles of 94° C. for 15 seconds, 60° C. for 30 seconds, 68° C. for 1 minute, with a final step of 68° C. for 7 minutes.

The 293 base-pair product was filtration purified using a Multiscreen PCR plate (Millipore, Billerica, Mass.) and then sequenced in both forward and reverse directions using the ABI Prism Big Dye Terminator V3.1 Cycle Sequencing Kit and the ABI PRISM 3100 Genetic Analyzer (Applied Biosystems, Foster City Calif.) using the JAK2 sequence in GenBank accession number NM004972 as a reference.

Results:

JAK2 V617F mutant nucleic acid could be detected by bi-directional sequencing in mixtures containing lysate from only five HEL cells per milliliter of plasma (data not shown).

2. Simultaneous Analysis of Cells and Plasma from MPD Patients

Simultaneous analysis of plasma and peripheral blood cells from 30 patients with myeloproliferative disease was performed as follows.

Blood and plasma were collected and prepared by methods known in the art. Peripheral blood cells were isolated from whole blood. Plasma was prepared by centrifugation in tubes containing EDTA (ethylenediaminetetraacetic acid), and the plasma was stored at −70° C. until assayed.

RNA was isolated, amplified and bi-directionally sequenced as described above, in Example 1.

Results:

Because direct sequencing of plasma or cell samples as routinely performed in clinical laboratories does not reliably distinguish between hemizygosity and homozygosity, these classes were grouped. That is, samples having no or minimal wild-type sequencing trace at the nucleotide corresponding to the V617F mutation are termed “hemizygous/homozygous.”

All samples that tested positive for the V617F mutation in cells also tested positive for this mutation in plasma. However, hemizygosity/homozygosity at the mutant locus was more evident in sequences from plasma, whereas cells of the same patient displayed both mutant and wild-type sequences. Heterozygous plasma samples showed equally intense wild-type (“G”) and mutant (“T”) peaks, whereas the mutant “T” peak was less conspicuous in the cell sample. This suggests that the essentially cell-free plasma is enriched with tumor-specific nucleic acid, and that the cell samples, containing populations of both malignant and non-malignant cells (e.g., lymphocytes carrying wild-type alleles of JAK2), give little or poor information regarding homozygous and hemizygous cell populations.

3. Testing Plasma from 86 Myeloproliferative Disease Patients

Plasma samples from 86 different patients, suffering from various chronic myeloproliferative diseases, were tested for the presence of the JAK2 V617F mutation as described above. Patients were diagnosed with MPD based on standard clinical findings, cytogenetics and RT-PCR analysis. As a control, plasma from 31 normal individuals was also tested for the mutation.

The 86 patients expressed different symptoms for a variety of MPDs. The disease status and characteristics of the 86 patients at the time of plasma draw are outlined in Tables 1 and 2, below. The median age of this population was 61 years, and 56% were male. (Table 1). A previous history of other malignancy was noted for 21% of patients, and 50% of the patients presented with an enlarged spleen. Although significant increases in bone marrow blasts were seen for some patients, 88% of the patients had blasts less than 5%, and 93% has blasts less than 10%.

TABLE 1
Patient Characteristics

| | |
|---|---|
| Patient | Number of |
| characteristics | patients |

SEX

| | |
|---|---|
| male | 48 |
| female | 38 |

ETHNICITY

| | |
|---|---|
| Caucasian | 80 |
| African | 4 |
| American | |
| Hispanic | 2 |

MEDICAL HISTORY

| | |
|---|---|
| prior | 18 |
| malignancy | |
| prior therapy | 3 |
| Performance | 74 |
| Status (PS) 0-1 | |
| PS 2 | 5 |
| PS missing | 7 |
| Enlarged liver | 16 |
| Enlarged spleen | 43 |

DIAGNOSED DISEASE

| | |
|---|---|
| IMF | 39 |
| PV | 16 |
| ET | 8 |
| MPD-NC | 23 |

TABLE 2
Detailed Medical Statistics For 86 MPD Patients

| | Median | Minimum | Maximum |
|---|---|---|---|
| AGE | 61 | 25.00 | 85.00 |
| Bone Marrow (%): | | | |
| Cellularity | 60.33 | 5.00 | 100.00 |
| Blasts | 3.52 | 0.00 | 61.00 |
| Monocytes | 2.76 | 0.00 | 17.00 |
| Eosinophils | 2.12 | 0.00 | 24.00 |
| Basophils | 1.51 | 0.00 | 30.00 |
| Erythroid cells | 17.12 | 0.00 | 67.00 |
| Peripheral Blood: | | | |
| Hemaglobin (g/dL) | 11.02 | 6.60 | 19.00 |
| Platelets (×10⁹/L) | 299.67 | 8.00 | 1181.00 |
| White blood cells | 22.35 | 1.70 | 182.00 |
| (×10⁹/L) | | | |
| Blasts (%) | 2.79 | 0.00 | 73.00 |
| Lymphocytes (%) | 16.30 | 0.00 | 59.00 |
| Monocytes (%) | 5.31 | 0.00 | 35.00 |
| Eosinophils (5) | 2.65 | 0.00 | 42.00 |
| Basophils (%) | 1.35 | 0.00 | 10.00 |
| Blood urea nitrogen | 16.25 | 7.00 | 39.00 |
| (mg/dL) | | | |
| Creatine (mg/dL) | 0.99 | 0.60 | 3.00 |
| Bilirubin (mg/dL) | 0.77 | 0.10 | 5.00 |
| Lactic dehydrogenase | 1476.58 | 503.00 | 4610.00 |
| (U/L) | | | |
| Alanine | 28.90 | 11.00 | 115.00 |
| aminotransferase (U/L) | | | |

Results:

Again, samples having no or minimal wild-type sequencing trace at the nucleotide corresponding to the V617F mutation are termed “hemizygous/homozygous.”

Overall, 51% (44/86) of the patient samples tested positive for the V617F mutation. Of these, 43% (19/44) were hemizygous/homozygous, while 57% (25/44) showed both a JAK2 V617F mutant sequence and a wild-type JAK2 sequence. No V617F mutant sequence was detected in any of the 31 control samples. Results show that most patients with PV and IMF carry the mutation, while the majority of ET and MPD-NC patients do not. Results are outlined in Table 3, below.

TABLE 3
Results Of Plasma Screening For V617f
Mutation In 86 MPD Patients.

| | | Homozygous | | | | | |
|---|---|---|---|---|---|---|---|
| Diagnosed | Number of | or | | | | | |
| disease | patients | hemizygous | | Heterozygous | | wild-type | |
| IMF | 39 | 9 | (23%) | 13 | (33%) | 17 | (44%) |
| PV | 16 | 7 | (43.5%) | 7 | (43.5%) | 2 | (13%) |
| ET | 8 | 1 | (13%) | 1 | (13%) | 6 | (75%) |
| MPD-NC | 23 | 2 | (9%) | 4 | (17%) | 17 | (74%) |
| Totals | 86 | 19 | (22%) | 25 | (29%) | 42 | (49%) |

Using bidirectional direct sequencing, the V617F mutation was found in 56% (22/39) of IMF patients and 41% (9/22) of the V617F-positive IMF patients were hemizygous/homozygous (Table 3). The V617F mutation was found in a greater proportion of PV patients, 88% (14/16), and more importantly, 50% (7/14) of the V617F-positive patients were hemizygous/homozygous (Table 3). For ET patients, 25% (2/8) of patients had the JAK2 mutation, and half of them (½) were hemizygous/homozygous (Table 3). Another group of patients with chronic MPD not otherwise classified (MPD-NC) was examined for the JAK2 mutation. They were negative for Philadelphia chromosome by cytogenetics (including FISH), and negative for BCR-ABL by molecular studies. Most of these patients had Philadelphia-negative CML or chronic neutrophilic leukemia, and a few had chronic MPD/myelodysplastic disease. In this group, the V617F mutation was detected in 26% (6/23) of the patients, and one-third (2/6) were hemizygous/homozygous (Table 3).

4. Correlations Between Patient Symptoms/Survival Rate and the V617F Mutation.

The significance of differences in clinical characteristics between groups of patients with different states of zygosity for the V617F mutation were analyzed by chi-square or Kruskal-Wallis test for categorical data and t test for continuous data. Estimates of survival curves were calculated according to Kaplan-Meier product-limit method and were calculated from the time of referral to MDACC. Survival times were compared by means of the log-rank test.

Compared with heterozygous patients, hemizygous/homozygous V617F patients had significantly enlargement of the spleen (P=0.0001), a greater percentage of monocytes (P=0.03), a higher white blood cell count (P=0.001), higher levels of bilirubin (P=0.02), and were more likely to have a history of prior malignancy (P=0.047) than heterozygous or wild-type patients. (Table 4). These results suggests that patients hemizygous or homozygous for the V617F mutation have a more aggressive disease.

TABLE 4
Differences between patients with heterozygous
and hemi/homozygous V617F mutations

| | Median values for V617F patients | | |
|---|---|---|---|
| | Hetero- | Homozygous or | |
| Condition | zygous | Hemizygous | P value |
| Spleen enlargement, cm | 0.0 | 15.0 | .0001 |
| Bone marrow monocytes, % | 2.0 | 1.0 | .03 |
| White blood cells ×10⁹ | 7.2 | 15.9 | .001 |
| Bilirubin, mg/dL | 0.55 | 0.80 | .02 |
| Prior malignancy (not | 8% | 32% | .047 |
| median values) | | | |

Survival rates did not differ significantly between heterozygote and hemizygous/homozygote patients (P=0.20; data not shown). However, three correlations were noted between survival and the V617F mutant for some patients. First, in patients in the chronic phase with less than 20% blast count (84 patients), heterozygous patients had significantly better survival rates than wild-type patients (P=0.04), while hemizygous/homozygous patient survival rates were not statistically different than those of wild-type patients (P=0.50). That is, a higher percentage of wild-type patients died than heterozygous patients, while death rates for homozygous and hemizygous patients were comparable to those of wild-type patients.

Second, in patients younger than 65 years old, heterozygous, hemizygous or homozygous carriers of the V617F mutation seem to have better survival than patients without the mutation (wild-type patients) (P=0.05; n=54).

Third, patients with an unclassified myeloproliferative disease (MPD-NC) tended to have a more aggressive disease and a shorter survival than the patients with PV, ET and IMF, (P=0.02). Most of these patients had chronic neutrophelic leukemia or Ph-negative chronic myelogeneous leukemia. Rare patients had MPD with meylodysplastic features. Interestingly, MPD-NC patients with the V617F mutant (either heterozygous, hemizygous or homozygous) seemed to have better survival times than MPD-NC patients without the mutation (P=0.05).

In summary, acellular bodily fluid such as plasma is a reliable source for the detection of JAK2 mutations and should be used to provide information on whether patients have heterozygous, hemizygous or homozygous mutant cell lineages. Further, the JAK2 V617F mutation defines a clinically important group of MPD patients with better overall outcome and survival than those who do not carry the mutation.

All publications, patent applications, patents and other references mentioned herein are expressly incorporated by reference in their entirety, to the same extent as if each were incorporated by reference individually. In case of conflict, the present specification, including definitions, will control.

Although the present inventions have been described with reference to exemplary and alternative embodiments, workers skilled in the art will recognize that changes may be made in form and detail without departing from the spirit and scope of the invention. For example, although different exemplary and alternative embodiments may have been described as including one or more features providing one or more benefits, it is contemplated that the described features may be interchanged with one another or alternatively be combined with one another in the described exemplary embodiments or in other alternative embodiments. Because the technology of the present invention is relatively complex, not all changes in the technology are foreseeable. The present invention described with reference to the exemplary and alternative embodiments and set forth in the following claims is manifestly intended to be as broad as possible. For example, unless specifically otherwise noted, the claims reciting a single particular element also encompass a plurality of such particular elements.

What is claimed is:

1. A method comprising determining the presence or absence of one or more mutations in JAK2 nucleic acid from an acellular bodily fluid of a patient.

2. The method of claim 1, wherein the presence or absence of one or more mutations is determined relative to SEQ ID NO: 1.

3. The method of claim 1, wherein the one or more mutations affects kinase activity.

4. The method of claim 1, wherein the one or more mutations is located in a pseudokinase domain of JAK2.

5. The method of claim 1, wherein the presence or absence of one or more mutations is determined relative to SEQ ID NO:2.

6. The method of claim 1, wherein the acellular bodily fluid is plasma or serum.

7. The method of claim 1, wherein at least one of the mutations is at codon 617.

8. The method of claim 1, wherein at least one of the mutations causes a V617F amino acid change.

9. The method of claim 1, wherein the JAK2 nucleic acid is RNA.

10. The method of claim 1, wherein the patient has been diagnosed with a myeloproliferative disease prior to said determining step.

11. The method of claim 1, wherein the determining step comprises amplifying JAK2 nucleic acid from the acellular bodily fluid of the patient.

12. The method of claim 1, wherein the determining step comprises amplifying nucleic acid from acellular bodily fluid of the patient and hybridizing the amplified nucleic acid with an oligonucleotide probe that is capable of specifically detecting JAK2 nucleic acid under hybridization conditions.

13. The method of claim 1 further comprising, determining the proportion of mutant JAK2 nucleic acid to wildtype JAK2 nucleic acid in said fluid.

14. The method of claim 1 further comprising, determining if the JAK2 nucleic acid comprises mutant JAK2 nucleic acid and wild-type JAK2 nucleic acid.

15. The method of claim 1 wherein the determination of a JAK2 nucleic acid mutation is used to stratify an individual for prognostic or therapeutic purposes.

16. A method of treatment for a patient with a neoplastic disease comprising, determining the presence or absence of one or more mutations in JAK2 nucleic acid from an acellular bodily fluid of the patient, and treating the patient based on the determination.

17. The method of claim 16, wherein the neoplastic disease is a myeloproliferative disease.

18. The method of claim 16, wherein the patient is a polycythemia vera patient.

19. The method of claim 16, wherein the patient is an essential thrombocythemia patient.

20. The method of claim 16, wherein the patient is an idiopathic myelofibrosis patient.

21. The method of claim 16, wherein the patient has an unclassified myeloproliferative disease.

22. The method of claim 16, wherein said one or more mutations affects kinase activity.

23. The method of claim 16, wherein the presence or absence of one or more mutations is determined relative to SEQ ID NO: 1.

24. The method of claim 16, wherein the one or more mutations is in a pseudokinase domain of JAK2.

25. The method of claim 16, wherein the presence or absence of one or more mutations is determined relative to SEQ ID: 2.

26. The method of claim 16, wherein the one or more mutations is at codon 617.

27. The method of claim 16, wherein the one or more mutations causes a V617F amino acid change.

28. The method of claim 16, further comprising, determining the proportion of JAK2 mutant nucleic acid to wildtype JAK2 nucleic acid in the fluid and treating the patient based on the determination.

29. The method of claim 16, further comprising, determining if the JAK2 nucleic acid comprises mutant JAK2 nucleic acid and wild-type JAK2 nucleic acid, and treating the patient based on the determination.

30. The method of claim 16, wherein the acellular bodily fluid is plasma or serum.

31. The method of claim 16, wherein the determining step comprises amplifying JAK2 nucleic acid obtained from the acellular bodily fluid of the patient and sequencing the amplified nucleic acid.

32. The method of claim 16, wherein the determining step comprises amplifying nucleic acid obtained from the acellular bodily fluid of the patient and hybridizing the amplified nucleic acid with an oligonucleotide probe that is capable of specifically detecting the JAK2 nucleic acid under hybridization conditions.

33. The method of claim 16, wherein a treatment is administered, foregone or changed based on the determination.

34. The method of claim 16, wherein a JAK2 mutant allele is detected and the other allele is determined to be deleted.

35. A method of determining whether a patient diagnosed with a neoplastic disease has cells containing JAK2 mutant kinase activity, comprising determining the presence or absence of one or more mutations in JAK2 nucleic acid from an acellular bodily fluid of the patient.

36. The method of claim 35, wherein the neoplastic disease is a myeloproliferative disease.

37. The method of claim 36, wherein the myeloproliferative disease is polycythemia vera.

38. The method of claim 36, wherein the myeloproliferative disease is essential thrombocythemia.

39. The method of claim 36, wherein the myeloproliferative disease is idiopathic myelofibrosis.

40. The method of claim 36, wherein the myeloproliferative disease is an unclassified myeloproliferative disease.

41. A method for diagnosing a neoplastic disease comprising determining the presence or absence of one or more mutations in JAK2 nucleic acid from an acellular bodily fluid of a patient.

42. The method of claim 41, wherein the presence or absence of one or more mutations is determined relative to SEQ ID NO: 1.

43. The method of claim 41, wherein the presence or absence of one or more mutations is determined relative to SEQ ID NO:2.

44. The method of claim 41, wherein the one or more mutations affects kinase activity.

45. The method of claim 41, wherein the one or more mutations is in a pseudokinase domain.

46. The method of claim 41, wherein the one or more mutations include a mutation at codon 617 that does not encode valine.

47. The method of claim 41, further comprising, determining the proportion of mutant JAK2 nucleic acid to wildtype JAK2 nucleic acid and diagnosing the patient based on the determination.

48. The method of claim 41, further comprising, determining if the JAK2 nucleic acid comprises mutant JAK2 nucleic acid and wild-type JAK2 nucleic acid, and diagnosing the patient based on the determination.

49. The method of claim 41, wherein the one or more mutations causes a V617F amino acid change.

50. The method of claim 46, wherein the mutation at codon 617 that does not encode valine is V617F.

51. The method of claim 41, wherein the JAK2 nucleic acid comprises RNA.

52. The method of claim 41, wherein determining comprises reverse transcribing JAK2 RNA.

53. The method of claim 41, wherein determining comprises amplifying JAK2 nucleic acid.

54. The method of claim 53, further comprising hybridizing the amplified JAK2 nucleic acid with a oligonucleotide probe that is specific for the amplified JAK2 nucleic acid.

55. The method of claim 53, further comprising sequencing the amplified JAK2 nucleic acid.

56. The method of claim 41, wherein the acellular bodily fluid comprises plasma or serum.

57. The method of claim 41, wherein the neoplastic disease is a myeloproliferative disease.

58. The method of claim 57, wherein the myeloproliferative disease is polycythemia vera.

59. The method of claim 57, wherein the myeloproliferative disease is essential thrombocythemia.

60. The method of claim 57, wherein the myeloproliferative disease is idiopathic myelofibrosis.

61. The method of claim 57, wherein the myeloproliferative disease is a myeloproliferative disease not classified as polycythemia vera, essential thrombocythemia, or idiopathic myelofibrosis.

62. A method of determining a prognosis of an individual diagnosed with a neoplastic disease, said method comprising determining the presence or absence of one or more mutations in JAK2 nucleic acid in an acellular bodily fluid of the individual and using the mutation status to predict the clinical outcome for the individual.

63. The method of claim 62, wherein said neoplastic disease is selected from the group consisting of polycythemia vera, essential thrombocythemia, idiopathic myelofibrosis, and unclassified myeloproliferative disease.

64. The method of claim 62, wherein the presence or absence of one or more mutations is determined relative to SEQ ID NO: 1.

65. The method of claim 62, wherein the one or more mutations affect kinase activity.

66. The method of claim 62, wherein the one or more mutations is located in a pseudokinase domain of JAK2.

67. The method of claim 62, wherein the presence or absence of one or more mutations is determined relative to SEQ ID NO:2.

68. The method of claim 62, wherein the acellular bodily fluid is plasma or serum.

69. The method of claim 62, wherein the one or more mutations is at codon 617.

70. The method of claim 62, wherein the one or more mutations causes a V617F amino acid change.

71. The method of claim 62, wherein the JAK2 nucleic acid is RNA.

72. The method of claim 62, wherein the determining step comprises amplifying JAK2 nucleic acid from the acellular bodily fluid of the patient.

73. The method of claim 62, wherein the determining comprises amplifying nucleic acid obtained from acellular bodily fluid of the patient and hybridizing the amplified nucleic acid with an oligonucleotide probe that is capable of specifically detecting JAK2 nucleic acid under hybridization conditions.

74. The method of claim 62, further comprising, determining the proportion of mutant JAK2 nucleic acid to wildtype JAK2 nucleic acid in said fluid.

75. The method of claim 62, wherein the mutation status is hemizygous or homozygous mutant for JAK2.

76. The method of claim 62, wherein mutation status is combined with other clinical parameters to determine the clinical outcome for the individual.

77. The method of claim 76, wherein the other clinical parameters is selected from the group consisting of age and percent blast cell count.

78. The method of claim 62, wherein the clinical outcome is death.


SEQUENCE LISTING (6 sequences)

SEQ ID NO: 1 - DNA, 3652 bp, Homo sapiens
tttctcttctgcagaaaaagaggctcttcctcctcctcccgcgacggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctggggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccacc

SEQ ID NO: 2 - DNA, 879 bp, Homo sapiens
cacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctg

SEQ ID NO: 3 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 4 - protein, 293 aa, Homo sapiens
HKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGAL

SEQ ID NO: 5 - DNA, 22 bp, Artificial Sequence
  Description of Artificial Sequence Synthetic primer
gactacggtcaactgcatgaaa

SEQ ID NO: 6 - DNA, 20 bp, Artificial Sequence
  Description of Artificial Sequence Synthetic primer
ccatgccaactgtttagcaa